(19) United States
(12) Patent Application Publication
(10) Pub. No.: US 2005/0074444 A1
(43) Pub. Date: Apr. 7, 2005

(54) Coenzyme Q10 treatment for cystic fibrosis

(76) Inventors: Andreas M. Papas, Kingsport, TN (US); Konstantinos A. Papas, Jonesborough, TN (US); Warren Hopkins, Kinsport, TN (US); William Andrew Clark, Johnson City, TN (US)

Correspondence Address: SUSAN F. JOHNSTON;SUSAN F. JOHNSTON, ATTORNEY AT LAW, P.O. BOX 4449, JOHNSON CITY, TN, 37602-4449, US

(21) Appl. No.: 10/945,006

(22) Filed: Sep. 20, 2004

Related U.S. Application Data
(60) Provisional application No. 60/507,830, filed on Oct. 1, 2003.

(51) Int. Cl. A61K 38/43; A61K 31/59; A61K 31/015; A61K 31/355

(52) U.S. Cl. 424/94.1; 514/167; 514/690; 514/763; 514/458

(57) ABSTRACT

Disclosed herein is an aqueous emulsion of coenzyme Q10 and preferably vitamin E, beta-carotene, vitamin D, and vitamin K useful for treating complications of cystic fibrosis. The aqueous emulsion improves malabsorption and immunity against infection, and reduces oxidative stress and respiratory complications in cystic fibrosis.

### CROSS REFERENCES TO RELATED APPLICATIONS

This application claims the benefit of U.S. Provisional Application Ser. No. 60/507,830 filed Oct. 1, 2003.

### BACKGROUND OF THE INVENTION

Cystic fibrosis is characterized by viscid secretions in the respiratory tract, pancreas, gastrointestinal tract, sweat glands, and other exocrine tissues. Increased viscosity of these secretions makes them difficult to clear. These heavy secretions result in symptoms of digestive problems due to pancreatic enzyme insufficiency and reduced bile pool, decreased respiratory function, and often infertility. End-stage lung disease is the primary cause of death in cystic fibrosis. Primary treatment for respiratory complications has included use of antibiotics, bronchodilators, and airway clearance techniques to retard the progression of lung disease. Chronic pulmonary infections, and other infections, in cystic fibrosis cause serious oxidative stress due to increased production of free radicals. Thus, the cystic fibrosis patient has a great need for increased dietary and supplemental antioxidants and other nutrients.

The severity of each of the symptoms of cystic fibrosis is magnified by the additional insult of malabsorption of important nutrients and dietary components. Malabsorption in cystic fibrosis greatly increases the complications of cystic fibrosis such as lung disease due to the fact that a decreased level of antioxidants and nutrients leads to oxidative stress in the tissues. The antioxidant defenses of cystic fibrosis patients are weakened as evidenced by lower activity of the selenium-dependent, antioxidant enzyme, glutathione peroxidase (SeGSH-Px), very low blood level of beta-carotene and vitamin E at the low end or below the normal range. Malabsorption in cystic fibrosis creates difficulty in delivering severely needed fat-soluble nutrients and antioxidants to the patient, which are important for maintaining normal immune and neurological function.

The malabsorption profile in cystic fibrosis is different from the malabsorption in other diseases such as inflammatory bowel disease and in cholestasis due to the fact that malabsorption in cystic fibrosis is caused by a combination of decreased production of pancreatic enzymes and decreased secretion of bile salts from the liver. Therefore, the malabsorption profile in cystic fibrosis reflects two separate abnormalities of the digestive system. The malabsorption complication in cystic fibrosis has been treated by enzyme and nutritional supplementation. The current nutritional supplements often fail to overcome this particular malabsorption complication.

In light of the above, it is an object of the present invention to provide an improved nutritional supplement formulation and method of treatment for reducing oxidative stress and increasing immunity to respiratory infection in a cystic fibrosis patient. It is a further object to provide a formulation that delivers an optimum blend of nutrients for improving chronic and acute respiratory exacerbation associated with cystic fibrosis. Still further, it is an object of the present invention to provide a daily nutritional supplement that is better absorbed in a cystic fibrosis patient for maintenance, intervention, and retardation of the progression of respiratory disease in cystic fibrosis.

### SUMMARY OF THE INVENTION

The present invention includes an aqueous emulsion that serves as a nutritional supplement and as a treatment for cystic fibrosis. The total weight of the aqueous emulsion comprises
a) from about 80.0 to about 98.0 weight percent water,
b) from about 1.0 to about 10.0 weight percent lipophilic component including from about 10 to 100 weight percent coenzyme Q10, from 0 to about 90 weight percent vitamin E compounds, from 0 to about 70 weight percent beta-carotene, from 0 to about 0.5 weight percent vitamin D, and from 0 to about 0.5 weight percent vitamin K, and
c) from about 1.0 to about 10.0 weight percent pharmaceutically acceptable solubilizer, with the preferred solubilizer being D-alpha tocopheryl polyethylene glycol-1000 succinate.

The invention further includes a method for treating cystic fibrosis using a therapeutically effective amount of coenzyme Q10. Specifically, the invention includes a method of administering the aqueous emulsion to treat respiratory exacerbation, increase immunity against respiratory infection, and overcome malabsorption in cystic fibrosis.

### DETAILED DESCRIPTION

The present invention is based upon the inventors' finding that coenzyme Q10 is particularly beneficial in improving oxidative stress and resultant respiratory complications associated with cystic fibrosis disease. The present invention includes a method of ameliorating the complications of cystic fibrosis, particularly oxidative stress and respiratory exacerbation, by delivering a therapeutically effective amount of easily absorbable, solubilized coenzyme Q10, and preferably other particularly useful nutrients in cystic fibrosis, through the wall of the small intestinal via oral delivery of an aqueous emulsion of solubilized coenzyme Q10 to the patient. It is crucial that the fat-soluble coenzyme Q10 be solubilized in the water component of the emulsion to such an extent that it forms absorbable micelles.

The aqueous emulsion for administration to a cystic fibrosis patient in the present invention includes from about 80.0 to about 98.0, preferably between about 90.0 to about 95.0, weight percent water; from about 1.0 to about 10.0, preferably about 1.0 to about 6.0, and more preferably from about 2.0 to about 5.0, weight percent of an active lipophilic component; and from about 1.0 to about 10.0, preferably from about 3.0 to about 6.0, weight percent of a pharmaceutically acceptable solubilizer affective for solubilizing the lipophilic component into the water.

The lipophilic component of the aqueous emulsion is at least about 10.0 weight percent coenzyme Q10. The lipophilic component can include up to 90.0 weight percent of the alpha-, beta-, delta, and gamma-tocopherols, the alpha-beta-, delta-, and gamma-tocotrienols, and pharmaceutically suitable esters of these homologues. The lipophilic component may include up to about 70.0 weight percent of beta-carotene. Up to about 0.5 weight percent of the lipophilic component may be made up of other lipophilic compounds. Of the many other useful lipophilic compounds, the most beneficial compound for blending in the present cystic fibrosis formulation are vitamin D and vitamin K.

The preferred embodiment of the aqueous emulsion has a lipophilic component comprising from about 10.0 to about 60.0 weight percent coenzyme Q10; from about 30.0 to about 80.0 weight percent of the vitamin E compounds; about 5.0 to about 35.0 weight percent beta-carotene; up to about 0.5 weight percent vitamin D; and up to about 0.5 weight percent vitamin K.

Examples of suitable esters of vitamin E tocopherols and tocotrienols in the present emulsion include the succinate esters, polyethylene glycol succinate esters, acetates, linoleates, nicotinates, phosphates, palmitates, and combinations thereof. Preferred esters are succinates and acetates. The use of non-esterified compounds is preferred in the present composition since they would not require the same amount of enzymatic cleaving for absorbability.

In addition to being useful as a source of alpha-tocopherol, D-alpha tocopheryl polyethylene glycol-1000 succinate, known as “vitamin E TGPS”, is the preferred solubilizer in the present invention. Dietary concentrations of vitamin E TPGS as a source of alpha-tocopherol in nutritional supplements are much lower than a concentration sufficient for solubilizing the present lipophilic component in water. It should be clear that when vitamin E TPGS is used as the solubilizing agent comprising between 1 to 10 weight percent of the weight of the present aqueous emulsion, vitamin E TPGS is not considered to be part of the weight of the lipophilic component of the emulsion since the lipophilic component is formed by the fat-soluble compounds that are solubilized by the solubilizer, i.e. vitamin E TPGS.

TPGS is a water-soluble form of natural-source vitamin E prepared by esterifying d-alpha-tocopheryl acid succinate with polyethylene glycol 1000 and is available from Eastman Chemical Company. Vitamin E TPGS has a chemical formula of C₃₃O₅H₅₄(CH₂CH₂O)ₙ, where “n” represents the number of polyethylene oxide moieties attached to the acid group of crystalline d-alpha tocopheryl acid succinate. Vitamin E TPGS is the preferred solubilizer in the present invention not only because it miscellizes the fat-soluble component for intestinal absorption, but also increases the cellular absorption of the miscellized lipophiles much better than other solubilizers. The unique properties of vitamin E TPGS are particularly beneficial in cystic fibrosis where both the bile salt solubilization and the enzymatic cleaving process of fat-soluble nutrients is reduced. The increased absorption of the lipophilic component of the formulation provided by vitamin E TPGS is particularly beneficial in cystic fibrosis since the harmful symptoms of the disease are magnified by oxidative stress due to malabsorption of fat-soluble nutrients.

Other pharmaceutically acceptable solubilizers suitable for use in the present invention include glycerin fatty acid esters (monoglycerides), acetic acid esters of monoglycerides (acetylated monoglyceride), lactic acid esters of monoglycerides (lactylated monoglyceride), citric acid esters of monoglycerides, succinic acid esters of monoglycerides, diacetyl tartaric acid esters of monoglycerides, polyglycerol esters of fatty acids (polyglycerol ester), polyglycerol polyricinoleate, sorbitan esters of fatty acids (sorbitan ester), propylene glycol esters of fatty acids (pg ester), sucrose esters of fatty acids (sugar ester), sodium stearoyl-2-lactylate, calcium stearoyl-2-lactylate, lecithin, enzyme digested lecithin/enzyme treated lecithin, polysorbate 20, polysorbate 60, polysorbate 80, cetearyl alcohol, cetyl alcohol, olive oil peg 7 esters, cetyl esters, and behenyl alcohol.

As used herein, the term “vitamin D” includes analogs of vitamin D as well as the various derivates of vitamin D including, for example, vitamin D1 (a molecular compound of lumisterol and vitamin D2), vitamin D2 (calciferol, ergocalciferol), vitamin D3 (cholecalciferol, activated 7-dehydrocholesterol), vitamin D4 (22:23-dihydroergocalciferol), and calcitriol (1,25-dihydrocholecalciferol). The preferred forms of vitamin D in the aqueous emulsion is vitamin D2 and vitamin D3.

The aqueous emulsion of the present invention is preferably made by melt blending the compounds comprising the lipophilic component with the solubilizer to form a lipid blend. Thereafter, the lipid blend is contacted with water to form about an 80 to about a 99 weight percent aqueous mixture. The mixture is intimately mixed for a period of time sufficient to form an emulsion that is stable at room temperature.

Examples of other antioxidants suitable for use in the present emulsion include butylated hydroxytoluene; butylated hydroxyanisole; propyl gallate; dodecylgallate; tert-butylhydroquinone; ethoxyquin; probucol; vitamin A as retinal; retinoic acid and retinyl acetate; alpha carotenes and gamma carotenes; other carotenoids including astaxanthin, lutein, lycopene, and zeaxanthin; ascorbic acid (vitamin C) and its calcium or sodium salts and ascorbyl palmitate; ubiquinols other than Coenzyme Q10; antioxidant enzymes such as glutathione and superoxide dismutase and their cofactors such as selenium (including selenomethionine and other forms), managanese, copper etc.; alpha-lipoic acid and dihydrolipoic acid; N-acetylcysteine and other amino acids with antioxidant activity such as cysteine; isoflavonoids and other phyotochemicals with antioxidant properties including including daidzin; genistein; quercetin; morin; curcumin; apigenin; sesamol; chlorogenic acid; fisetin; ellagic acid; quillaia saponin; capsaicin; ginsenoside; silymarin; kaempferol; ginkgetin; bilobetin; isoginkgetin; isorhamnetin; herbimycin; rutin; bromelain; levendustin A; and erbstatin; B vitamins (especially folic acid and biotin); amino acids especially sulfur containing amino acids; fatty acids especially omega-3 fatty acids; sugars; carnitine and acetyl-L-carnitine.

The aqueous emulsion for treating cystic fibrosis formed according to the present invention has a total weight distribution of about 80.0 to about 98.0 weight percent water; about 0.05 to about 10.0 weight percent coenzyme Q10; up to about 10.0 weight percent of the vitamin E compounds; up to about 2.5 weight percent beta-carotene; up to about 0.005 weight percent vitamin D; up to about 0.05 weight percent vitamin K; and from about 1.0 to about 15.0 weight percent solubilizer.

The preferred aqueous emulsion for treating cystic fibrosis formed according to the present invention has a total weight distribution of about 85.0 to about 95.0 weight percent water, about 0.1 to about 5.0 weight percent coenzyme Q10, from about 1.0 to about 5.0 weight percent the vitamin E compounds, from about 0.1 to about 1.0 weight percent beta-carotene; from about 0.0001 to about 0.001 weight percent vitamin D; from about 0.001 to about 0.01 weight percent vitamin K, and from about 3.5 to about 7.0 weight percent solubilizer, with the preferred solubilizer being D-alpha tocopheryl polyethylene glycol-1000 succinate (vitamin E TPGS).

The aqueous emulsion of the present invention is preferably delivered orally as an emulsion or as a capsule, such as a gelatin capsule, filled with the aqueous emulsion. The invention includes a method of treating a cystic fibrosis patient with an oral dosage of the aqueous emulsion formulated to deliver a sufficient dosage of coenzyme Q10 and other nutrients to treat chronic or acute respiratory conditions associated with cystic fibrosis. Respiratory complications of cystic fibrosis improved by treatment with the present emulsion include viscous secretions, inflammation, infection, scarring, mucus plugging, and other similar complications. An acute respiratory exacerbation of a respiratory complication, as used herein, refers to a sudden onset or spike in these complications whereas a chronic respiratory complication is a continuous baseline condition for a patient at his particular disease state.

The invention comprises a method of increasing the cystic fibrosis patient's immunity against respiratory infection by administering an effective amount of the emulsion on a daily basis to reduce oxidative stress. The effective amount of the present emulsion for administration in the present invention is an amount sufficient to deliver between about 0.1 to about 100 mg coenzyme Q10 per kg body weight per day. The preferred amount of coenzyme Q10 delivered in the present invention is between about 1 to about 50, and more preferably between about 1 to about 25 mg per kg of body weight per day.

Although treatment with coenzyme Q10 alone has been found to be effective in treating cystic fibrosis in the present invention, the most effective embodiment of the present invention combines coenzyme Q10 with the other fat-soluble compounds as stated above as the preferred lipophilic component in a vitamin E TPGS solubilized emulsion so that the malabsorption profile of cystic fibrosis can be particularly and specifically overcome. Thus, treatment of a cystic fibrosis patient with the preferred composition of the present invention provides therapy for overcoming malabsorption and provides therapy for preventing or reducing oxidative stress and respiratory exacerbation.

The invention has been described in detail with particular reference to preferred embodiments thereof, but it will be understood that variations and modifications can be effected within the spirit and scope of the invention.

1. A nutritional supplement for treating cystic fibrosis, said nutritional supplement comprising:
a) from about 80.0 to about 98.0 weight percent water,
b) from about 1.0 to about 10.0 weight percent lipophilic component including from about 10 to 100 weight percent coenzyme Q10;
from 0 to about 90 weight percent vitamin E compound selected from the group consisting of alpha-tocopherol, an ester of alpha-tocopherol, beta-tocopherol, an ester of beta-tocopherol, delta-tocopherol, an ester of delta-tocopherol, gamma-tocopherol, an ester of gamma-tocopherol, alpha-tocotrienol, an ester of alpha-tocotrienol, beta-tocotrienol, an ester of beta-tocotrienol, delta-tocotrienol, an ester of delta-tocotrienol, gamma-tocotrienol, an ester of gamma-tocotrienol, and a combination thereof,
from 0 to about 70 weight percent beta-carotene,
from 0 to about 0.5 weight percent vitamin D, and
from 0 to about 0.5 weight percent vitamin K, and
c) from about 1.0 to about 10.0 weight percent pharmaceutically acceptable solubilizer suitable for solubilizing said lipophilic component into said water, wherein the sum of said weight percentages equals a total of 100 weight percent.

2. The nutritional supplement according to claim 1 wherein said lipophilic component comprises from about 10 to about 60 weight percent said coenzyme Q10, from about 30 to about 80 weight percent said vitamin E compounds, from about 5 to about 35 weight percent beta-carotene, from 0 to about 0.5 weight percent vitamin D, and from 0 to about 0.5 weight percent vitamin K.

3. The nutritional supplement according to claim 1 wherein each of said esters in said lipophilic component is selected from the group of esters consisting of succinates, acetates, nicotinates, phosphates, linoleates, palmitates and a combination thereof.

4. The nutritional supplement according to claim 1 wherein said aqueous emulsion comprises from about 90.0 to about 95.0 weight percent water, from about 2.0 to about 5.0 weight percent lipophilic component, and from about 3.0 to about 6.0 weight percent solubilizer.

5. The nutritional supplement according to claim 1 wherein said solubilizer is D-alpha tocopheryl polyethylene glycol-1000 succinate.

6. An aqueous emulsion for the treatment of cystic fibrosis, said aqueous emulsion comprising:
a) from about 80.0 to about 98.0 weight percent water;
b) from about 0.05 to about 10.0 weight percent coenzyme Q10;
c) from 0 to about 10.0 weight percent vitamin E compounds selected from the group consisting of alpha-tocopherol, an ester of alpha-tocopherol, beta-tocopherol, an ester of beta-tocopherol, delta-tocopherol, an ester of delta-tocopherol, gamma-tocopherol, an ester of gamma-tocopherol, alpha-tocotrienol, an ester of alpha-tocotrienol, beta-tocotrienol, an ester of beta-tocotrienol, delta-tocotrienol, an ester of delta-tocotrienol, gamma-tocotrienol, an ester of gamma-tocotrienol, and a combination thereof;
d) from 0 to about 2.5 weight percent beta-carotene;
e) from 0 to about 0.005 weight percent vitamin D;
f) from 0 to about 0.05 weight percent vitamin K; and
g) from about 1.0 to about 15.0 weight percent pharmaceutically acceptable solubilizer suitable from solubilizing each of said coenzyme Q10, vitamin E compounds, beta-carotene, vitamin D, and vitamin K into said water,
wherein the sum of said weight percentages equals a total of 100 weight percent.

7. The aqueous emulsion according to claim 6 comprising
a) from about 85.0 to about 95.0 weight percent said water;
b) from about 0.1 to about 5.0 weight percent said coenzyme Q10;
c) from about 1.0 to about 5.0 weight percent said vitamin E compounds;
d) from about 0.1 to about 1.0 weight percent said beta-carotene;
e) from about 0.0001 to about 0.001 weight percent said vitamin D;
f) from about 0.001 to about 0.01 weight percent said vitamin K; and
g) from about 3.5 to about 7.0 weight percent said solubilizer.

8. The aqueous emulsion according to claim 6 wherein each said ester in said lipophilic component is selected from the group consisting of succinates, acetates, nicotinates, phosphates, linoleates, palmitates and a combination thereof.

9. The aqueous emulsion according to claim 6 wherein said solubilizer is D-alpha tocopheryl polyethylene glycol-1000 succinate.

10. A gelatin capsule filled with the aqueous emulsion according to claim 6.

11. A method of treating a respiratory complication of cystic fibrosis, said method comprising:
orally administering to the patient a therapeutically effective amount of an aqueous emulsion comprising
a) from about 80.0 to about 98.0 weight percent water;
b) from about 1.0 to about 10.0 weight percent lipophilic component including
from about 10 to 100 weight percent coenzyme Q10,
from 0 to about 90 weight percent vitamin E compounds selected from the group consisting of alpha-tocopherol, an ester of alpha-tocopherol, beta-tocopherol, an ester of beta-tocopherol, delta-tocopherol, an ester of delta-tocopherol, gamma-tocopherol, an ester of gamma-tocopherol, alpha-tocotrienol, an ester of alpha-tocotrienol, beta-tocotrienol, an ester of beta-tocotrienol, delta-tocotrienol, an ester of delta-tocotrienol, gamma-tocotrienol, an ester of gamma-tocotrienol, and a combination thereof,
from 0 to about 70 weight percent beta-carotene,
from 0 to about 0.5 weight percent vitamin D,
from 0 to about 0.5 weight percent vitamin K; and
c) from about 1.0 to about 10.0 weight percent pharmaceutically acceptable solubilizer suitable for solubilizing said lipophilic component into said water.

12. The method according to claim 11 wherein said respiratory complication is characterized by an acute exacerbation of said complication.

13. The method according to claim 11 wherein said respiratory complication is in a chronic state.

14. The method according to claim 11 wherein said solubilizer is D-alpha tocopheryl polyethylene glycol-1000 succinate.

15. The method according to claim 11 wherein each said ester in said lipophilic component is selected from the group consisting of succinates, acetates, nicotinates, phosphates, linoleates, palmitates and a combination thereof.

16. The method according to claim 11 wherein said aqueous emulsion comprises
from about 90.0 to about 95.0 weight percent water, from about 2.0 to about 5.0 weight percent lipophilic component, and from about 3.0 to about 6.0 weight percent solubilizer.

17. The method according to claim 11 wherein said lipophilic component comprises from about 10 to about 60 weight percent said coenzyme Q10, from about 30 to about 80 weight percent said vitamin E compounds, from about 5 to about 35 weight percent beta-carotene, from 0 to about 0.5 weight percent vitamin D, and from 0 to about 0.5 weight percent vitamin K.

18. A method of increasing a cystic fibrosis patient's immunity against respiratory infection, comprising orally administering a sufficient amount of an aqueous emulsion adapted for delivering a therapeutically effective amount of coenzyme Q10 to the patient.

19. The method according to claim 18 wherein the amount of aqueous emulsion administered is sufficient for delivering from between about 0.1 to about 100 mg coenzyme Q10 per kg body weight per day.

20. The method according to claim 19 wherein the amount of aqueous emulsion administered is sufficient for delivering from between about 1 to about 50 mg coenzyme Q10 per kg body weight per day.

21. The method according to claim 18 wherein said aqueous emulsion comprises,
a) from about 80.0 to about 98.0 weight percent water,
b) from about 1.0 to about 10.0 weight percent lipophilic component including
from about 10 to 100 weight percent coenzyme Q10,
from 0 to about 90 weight percent vitamin E compound selected from the group consisting of alpha-tocopherol, an ester of alpha-tocopherol, beta-tocopherol, an ester of beta-tocopherol, delta-tocopherol, an ester of delta-tocopherol, gamma-tocopherol, an ester of gamma-tocopherol, alpha-tocotrienol, an ester of alpha-tocotrienol, beta-tocotrienol, an ester of beta-tocotrienol, delta-tocotrienol, and ester of delta-tocotrienol, gamma-tocotrienol, an ester of gamma-tocotrienol, and a combination thereof,
from 0 to about 70 weight percent beta-carotene,
from 0 to about 0.5 weight percent vitamin D,
from 0 to about 0.5 weight percent vitamin K, and
c) from about 1.0 to about 10.0 weight percent pharmaceutically acceptable solubilizer suitable for solubilizing said lipophilic component into said water, wherein the sum of said weight percentages equals a total of 100 weight percent.

22. The method according to claim 21 wherein said pharmaceutically acceptable solubilizer is D-alpha tocopheryl polyethylene glycol-1000 succinate.

23. A method of treating malabsorption of fat-soluble nutrients in cystic fibrosis, said method comprising:
orally administering to the patient a therapeutically effective amount of an aqueous emulsion comprising
a) from about 80.0 to about 98.0 weight percent water,
b) from about 1.0 to about 10.0 weight percent lipophilic component including
from about 10 to 100 weight percent coenzyme Q10,
from 0 to about 90 weight percent vitamin E compounds selected from the group consisting of alpha-tocopherol, an ester of alpha-tocopherol, beta-tocopherol, an ester of beta-tocopherol, delta-tocopherol, an ester of delta-tocopherol, gamma-tocopherol, an ester of gamma-tocopherol, alpha-tocotrienol, an ester of alpha-tocotrienol, beta-tocotrienol, an ester of beta-tocotrienol, delta-tocotrienol, an ester of delta-tocotrienol, gamma-tocotrienol, an ester of gamma-tocotrienol, and a combination thereof,
from 0 to about 70 weight percent beta-carotene,
from 0 to about 0.5 weight percent vitamin D,
from 0 to about 0.5 weight percent vitamin K, and
c) from about 1.0 to about 10.0 weight percent pharmaceutically acceptable solubilizer suitable for solubilizing said lipophilic component into said water.

24. The method according to claim 23 wherein said solubilizer is D-alpha tocopheryl polyethylene glycol-1000 succinate.

25. The method according to claim 23 wherein each said ester in said lipophilic component is selected from the group consisting of succinates, acetates, nicotinates, phosphates, linoleates, palmitates and a combination thereof.

26. The method according to claim 24 wherein said aqueous emulsion comprises
from about 90.0 to about 95.0 weight percent water, from about 1.0 to about 6.0 weight percent lipophilic component, and from about 3.0 to about 6.0 weight percent solubilizer.

27. The method according to claim 23 wherein said lipophilic component comprises from about 10 to about 60 weight percent coenzyme Q10, from about 30 to about 80 weight percent said vitamin E compounds, from about 5 to about 35 weight percent beta-carotene, from 0 to about 0.5 weight percent vitamin D, and from 0 to about 0.5 weight percent vitamin K.
